(19) United States
(12) Patent Application Publication
(10) Pub. No.: US 2018/0221369 A1
(43) Pub. Date: Aug. 9, 2018

(54) COMPOSITIONS AND METHODS FOR TREATING FIBROSING DISORDERS AND CANCER

(71) Applicant: Cedars-Sinai Medical Center, Los Angeles, CA (US)

(72) Inventors: Andrzej Ptasznik, Los Angeles, CA (US); Stephen Pandol, Los Angeles, CA (US); Hung Pham, Santa Monica, CA (US)

(73) Assignee: Cedars-Sinai Medical Center, Los Angeles, CA (US)

(21) Appl. No.: 15/506,081

(22) PCT Filed: Sep. 2, 2015
(86) PCT No.: PCT/US15/48202

§ 371 Date: Feb. 23, 2017

Related U.S. Application Data
(60) Provisional application No. 62/044,675, filed on Sep. 2, 2014.

(51) Int. Cl. A61K 31/506; A61K 45/06; A61P 35/04

(52) U.S. Cl. CPC A61K 31/506; A61K 45/06; A61K 31/713; A61K 2300/00; A61P 35/04

(57) ABSTRACT

The invention provides methods for treating fibrosis, chronic inflammation, chronic pancreatitis, cancer or inflammatory myofibroblastic tumors (IMTs) in a subject in need thereof. The methods include providing a composition comprising a Lyn inhibitor and administering an effective amount of the composition to the subject to treat fibrosis, chronic inflammation, chronic pancreatitis, cancer or inflammatory myofibroblastic tumors (IMTs). fibrosis, chronic inflammation, chronic pancreatitis, cancer or inflammatory myofibroblastic tumors (IMTs).

### GOVERNMENT RIGHTS

The invention was made with government support under Grant Nos. CA163200-01A1 and AA011999 awarded by the National Institutes of Health. The government has certain rights to the invention.

### FIELD OF INVENTION

Provided herein are compositions and methods for treating, inhibiting, reducing and/or promoting prophylaxis of fibrosis, chronic inflammation, chronic pancreatitis, cancer or inflammatory myofibroblastic tumors (IMTs) in subject in need thereof. The compositions comprise inhibitors of Lyn tyrosine kinase.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Chronic pancreatitis (CP) is a persistent inflammation of the pancreas that leads to damage of parenchyma, gradual fibrotic replacement of the gland, and serious complications, including loss of exocrine and endocrine function and cancer. Although it is proven that activated, α-smooth muscle actin-(α-SMA)-positive, stellate cells play a key role in infiltration and replacement of pancreatic parenchyma, no specific signaling molecules directly involved in regulating migration of these cells within the pancreas of CP has been identified. No effective anti-inflammatory and anti-fibrotic therapies exist to date due to the limited understanding of the key driving signaling mechanisms in pancreatic stellate cells. Parenchyma is also replaced with stellate cells during chronic inflammation of liver, and other organs, for which the therapeutic targets are unknown, as well.

Stromal cell-derived factor-1 (SDF-1) chemokine and its receptor, CXCR4, are known as the master regulators of movement of stem/progenitor cells, leukocytes, and other cells, within their local tissue microenvironment, and CXCR4 expression is increased in inflamed tissues. Lyn kinase is involved in chemo-attractant signaling and is required for CXCR4-mediated chemotaxis of hematopoietic cells and macrophages. However, the role of Lyn kinase in migration of stellate cells in inflammation of the pancreas or any other fibrotic organ during chronic inflammation has not been reported to date. The inventors provide evidence that Lyn kinase activity is selectively increased in stellate myofibroblastic cells of pancreatic microenvironment in CP, which leads to hyperactive CXCR4-mediated migration of the activated stellate cells within pancreas.

### SUMMARY OF THE INVENTION

Accordingly, provided herein are compositions comprising inhibitors of Lyn kinase such as Lyn kinase antagonist polypeptide, Lyn kinase-specific inhibitory nucleic acid, Lyn kinase-specific antagonist antibody or an antigen binding fragment thereof, Lyn kinase-specific small molecule, that inhibit or cause or facilitate a qualitative or quantitative inhibition, decrease, or reduction in one or more processes, mechanisms, effects, responses, functions, activities, or pathways mediated by Lyn kinase, and methods comprising such Lyn kinase inhibitors.

In some embodiments, provided herein are Lyn kinase-specific RNA interference agents that specifically target Lyn kinase and can be used for the inhibition of expression of Lyn kinase. In some embodiments, the RNA interference agent or siRNA is a Lyn kinase-specific double stranded RNA (dsRNA). In some embodiments, the RNA interference agent or siRNA is a Lyn kinase-specific small hairpin RNA (shRNA). In some aspects, the Lyn kinase-specific inhibitor is Bafetinib.

Provided herein is a method for treating, inhibiting, reducing the severity of and/or promoting prophylaxis of a disease-state in a subject in need thereof. The method comprises, consists of or consists essentially of providing a composition comprising a Lyn kinase inhibitor and administering an effective amount of the composition to the subject so as to treat, inhibit, reduce the severity of and/or promote prophylaxis of the disease state. In various embodiments, the disease-state is fibrosis, chronic inflammation, chronic pancreatitis, cancer or inflammatory myofibroblastic tumors (IMTs). In some embodiments, the Lyn kinase inhibitor is a siRNA specific for the Lyn kinase. In an embodiment, Lyn kinase inhibitor is Bafetinib. In one embodiment, the subject is human. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of chronic pancreatitis in a subject in need thereof. The method comprises, consists of or consists essentially of providing a composition comprising a Lyn kinase inhibitor and administering an effective amount of the composition to the subject so as to treat, inhibit, reduce the severity of and/or promote prophylaxis of chronic pancreatitis in the subject. In some embodiments, the Lyn kinase inhibitor is a siRNA specific for Lyn kinase. In an embodiment, the Lyn kinase inhibitor is Bafetinib. In one embodiment, the subject is human. In some embodiments, the methods may further comprise administering existing treatments for chronic pancreatitis and managing chronic pancreatitis using, for example, pharmacologic alleviation of abdominal pain, pharmacologic restoration of digestion and absorption, endoscopic treatments, surgical therapies, dietary care or combinations thereof. The composition comprising the Lyn kinase inhibitor and existing therapies may be administered sequentially or simultaneously. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of fibrosis in a subject in need thereof. The method comprises, consists of or consists essentially of providing a composition comprising a Lyn kinase inhibitor and administering an effective amount of the composition to the subject so as to treat, inhibit, reduce the severity of and/or promote prophylaxis of fibrosis. In some embodiments, the Lyn kinase inhibitor is a siRNA specific for Lyn kinase. In an embodiment, the Lyn kinase inhibitor is Bafetinib. In various embodiments of the methods described herein, fibrosis is any one or more of fibrosis of the pancreas, cystic fibrosis, mediastinal fibrosis, myelofibrosis, retroperitoneal fibrosis, progressive massive fibrosis, nephrogenic systemic fibrosis, Crohn's Disease, Keloid, Scleroderma/systemic sclerosis, Arthrofibrosis, Peyronie's disease, Dupuytren's contracture, adhesive capsulitis, fibrosis of the liver, fibrosis of the lung, fibrosis of the intestine (for example, in Crohn's disease), fibrosis of the heart, or combinations thereof. In some embodiments, the method comprises, consists of or consists essentially of providing an agent that inhibits Lyn kinase and administering an effective amount of the agent to the subject.

In some embodiments, secondary organ fibrosis is a side-effect of radiation therapy. Accordingly, in one embodiment, the subject being treated for fibrosis using the Lyn kinase inhibitors is a subject that has undergone or is undergoing radiation therapy for a cancer in the first organ and shows or may show symptoms or other findings of fibrosis in a second organ, wherein the first and second organs are different. In some embodiments, the subject being treated for fibrosis is a subject that has undergone or is undergoing radiation therapy for treatment of cancer the first organ and shows symptoms or other findings of fibrosis at another location in the same organ. In some embodiments, the subject being treated for fibrosis is a subject undergoing radiation for treatment of cancer and shows or may show symptoms or other findings of fibrosis at the site of the cancer. In some embodiments, the subject being treated for fibrosis is a subject that has been exposed to radiation due to occupational reasons such as administrators of radiation therapy, military personnel and the like.

The methods for treating, inhibiting, reducing the severity of or promoting prophylaxis of fibrosis may further comprise administering existing therapies for organ-specific fibrosis in conjunction with (simultaneously or sequentially) Lyn kinase inhibitors.

In an exemplary embodiment, the methods may further comprise pharmacologic alleviation of abdominal pain, pharmacologic restoration of digestion and absorption, endoscopic treatments, surgical therapies, dietary care or combinations thereof for fibrosis in the pancreas.

In another exemplary embodiment, the methods may further comprise oxygen therapy, prednisone, azathioprine and/or N-acetylcysteine for lung fibrosis.

In an additional exemplary embodiment, the methods may further comprise administering angiotensin converting enzyme inhibitor or Lisinopril for myocardial fibrosis.

Provided herein are methods for treating, inhibiting, reducing the severity of or promoting prophylaxis of chronic inflammation in a subject in need thereof. The method comprises, consists of or consists essentially of providing a composition comprising a Lyn kinase inhibitor and administering an effective amount of the composition to the subject so as to treat, inhibit, reduce the severity of and/or promote prophylaxis of chronic inflammation in the subject. In some embodiments, the Lyn kinase inhibitor is a siRNA specific for Lyn kinase. In an embodiment, the Lyn kinase inhibitor is Bafetinib. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of disease-states associated with hyperactive Lyn kinase activity in cells (for example, in activated stellate cells) and/or disease-states associated with activated stellate cell proliferation and/or migration. The method comprises, consists of or consists essentially of providing a composition comprising a Lyn kinase inhibitor and administering an effective amount of the composition to the subject so as to treat, inhibit, reduce the severity of and/or promote prophylaxis of disease-states associated with hyperactive Lyn kinase activity in cells and/or disease-states associated with the activated stellate cell proliferation and/or migration. In some embodiments, the Lyn kinase inhibitor is a siRNA specific for Lyn kinase. In an embodiment, the Lyn kinase inhibitor is Bafetinib. In exemplary embodiments, the disease-states include chronic pancreatitis, chronic inflammation, fibrosis, cancer or a combination thereof. In some embodiments, the method comprises, consists of or consists essentially of providing an agent that inhibits Lyn kinase and administering an effective amount of the agent to the subject.

Provided herein is a method for treating cancer or treating or preventing cancer metastasis in a subject in need thereof. The method comprises, consists of or consists essentially of providing a composition comprising and Lyn kinase inhibitor and administering an effective amount of the composition to the subject so as to treat or prevent cancer metastasis in the subject. In some embodiments, the Lyn kinase inhibitor is a siRNA specific for Lyn kinase. In an embodiment, the Lyn kinase inhibitor is Bafetinib. In one embodiment, the subject is human. In various embodiments, the cancer is associated with activated stellate cells in an organ, including but not limited to activated stellate cells in the pancreas, lung, liver, bone, respiratory tract, urinary tract, lymphoid organs or neurons. Activated stellate cells are the names applied to activated myofibroblastic cells in the pancreas and liver. However, the name activated myofibroblastic cell is the general name given to these cells in other organs as well as the pancreas and liver. In some embodiments, the method comprises, consists of or consists essentially of providing an agent that inhibits Lyn kinase and administering an effective amount of the agent to the subject. In various embodiments, the method may further comprise for example, treating the subject with surgery, radiation therapy, or chemotherapy, or a combination thereof. The surgery, radiation therapy, or chemotherapy, or a combination thereof may be conducted before, during or after administering a therapeutically effective amount of the composition to the subject. In various embodiments of the methods described herein, the cancer is any one or more of lymphomas, sarcomas, brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer.

Provided herein are methods for treating, inhibiting, reducing the severity of or promoting prophylaxis of inflammatory myofibroblastic tumors in a subject in need thereof. The method comprises, consists of or consists essentially of providing a composition comprising a Lyn kinase inhibitor and administering an effective amount of the composition to the subject so as to treat, inhibit, reduce the severity of and/or promote prophylaxis of inflammatory myofibroblastic tumors in the subject. In some embodiments, the Lyn kinase inhibitor is a siRNA specific for Lyn kinase. In an embodiment, the Lyn kinase inhibitor is Bafetinib. In one embodiment, the subject is human. In various embodiments of the methods described herein the inflammatory myofibroblastic tumors are in any one or more of bladder, bone, breast, CNS tumor, colon tumor, eye (orbit), heart tumor, kidney tumor, liver tumor, lung tumor, lymph node, mediastinum, pancreas, salivary glands, small bowel, spleen and/or thyroid gland. In some embodiments, the method comprises, consists of or consists essentially of providing a Lyn kinase inhibitor and administering an effective amount of the inhibitor to the subject.

Also provided herein are methods for identifying inhibitors of Lyn kinase. The methods comprise contacting the Lyn kinase in Lyn kinase positive cells with a molecule of interest and determining whether the contact results in decreased proliferation and/or migration of activated stellate cells or myofibroblast. In various embodiments, a decrease in proliferation and/or migration of stellate cells or myofibroblast indicates that the molecule of interest is a Lyn kinase inhibitor. In an exemplary embodiment, the activated stellate cells are activated pancreatic stellate cells. In various embodiments, the Lyn kinase inhibitor is selected from the group consisting of a small molecule, a peptide, an antibody or a fragment thereof and a nucleic acid molecule. In some embodiments, the screening method comprises separately contacting each of a plurality of samples to be tested. In some embodiments, the plurality of samples comprises more than about 10⁴ samples. In some embodiments, the plurality of samples comprises more than about 5×10⁴ samples. In some embodiments, the stellate cells are activated stellate cells. In some embodiments, the activated stellate cells are obtained from patients with chronic pancreatitis. In some embodiments, the activated stellate cells are obtained from patients with pancreatic cancer. In some embodiments, myofibroblasts are obtained from human tissues of a fibrosing disorder are used. In some embodiments, myofibroblasts are obtained from human cancers.

### BRIEF DESCRIPTION OF FIGURES

Exemplary embodiments are illustrated in the referenced figures. It is intended that the embodiments and figures disclosed herein are to be considered illustrative rather than restrictive.

FIG. 1A-1F depict, in accordance with various embodiments of the present invention, increased Lyn kinase activity and CXCR4 receptor expression in activated α-SMA-positive stellate cells in human chronic pancreatitis. Pancreatic stellate cells were isolated from chronic pancreatitis patients (hCP) and normal donors (hNP). Subsequently, we performed Lyn and Src kinase assays (FIG. 1A, FIG. 1B), and Western blotting for Lyn kinase, Src, α-SMA (FIG. 1C) and total CXCR4 protein (FIG. 1D) in these cells. Kinase assays and Western blot analysis were normalized to the housekeeping gene GAPDH. FIG. 1E shows flow cytometry analysis of CXCR4 receptor expression on stellate cells of chronic pancreatitis (hCP) and normal subject (hNP). Cells were stained with or without (control) anti-human CXCR4 antibody followed by secondary antibodies and analysis. FIG. 1A to FIG. 1E are shown for the same pancreatitis and normal subject. We obtained the consistent results with the four different subjects (n=4). FIG. 1F shows representative fields for chronic pancreatitis and normal pancreas (n=6). Bar graph represents the morphometric and statistical analysis performed on 150 images per tissue group and indicates increased coexpression (arrows) of CXCR4 with α-SMA, and infiltration of CXCR4+/α-SMA+ cells into atrophic parenchyma in pancreatitis tissue (arrows, top left quadrant), which is in contrast to the intact parenchymal structures in normal pancreata (top right quadrant). This is consistent with Western blot showing increased coexpression of α-SMA with CXCR4 in the pancreatitis patient (hCP) (FIG. 1C, FIG. 1D, and FIG. 1E).

FIG. 2A-2H depict, in accordance with various embodiments of the present invention, the robust differential chemotactic response to stimulation or inhibition of CXCR4/Lyn kinase signaling between activated stellate cells of chronic pancreatitis (hCP) and normal donors (hNP). In FIG. 2A control Western blotting were performed 48 h after electroporation with siRNA. In FIG. 2B cells were stimulated with control buffer or 0.025 μM SDF-1 for 2 h. The result shown is representative of experiments with 4 different cell donors. In FIG. 2C, FIG. 2D and FIG. 2E, cells were treated with AMD3100, siRNA or INNO-406, as indicated, and subsequently chemotaxis assays were performed in SDF-1 stimulated or unstimulated cells (n=4). FIG. 2F shows control cell counts in growing chronic pancreatitis (hCP) versus normal (hNP) stellate cells, and control MTT viability assays were performed 24 h and 72 h after electroporation with siRNA (n=6). FIG. 2G shows that Cerulein-induced pancreatic fibrosis is ameliorated by treatment with INNO-406. Hematoxilin/eosin or Picro-Sirius Red stains denotes fibrosis; connective tissue is shown in dark grey. Cerulein and INNO-406 decrease pancreatic fibrosis compared to cerulean alone (untreated: 2.00+/−0.84%, cerulein: 16.39+/−3.08%, cerulein and INNO-406: 7.50+/−4.77%). Saline-treated mice were used as negative control (untreated). 10× magnification. FIG. 2H shows the same areas as FIG. 2G but with 20× magnification.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 March 24, 332(6162):323-7.

For references on pediatrics, see Schwartz et al., The 5-Minute Pediatric Consult 4th ed., Lippincott Williams & Wilkins, (Jun. 16, 2005); Robertson et al., The Harriet Lane Handbook: A Manual for Pediatric House Officers 17^{th} ed., Mosby (Jun. 24, 2005); and Hay et al., Current Diagnosis and Treatment in Pediatrics (Current Pediatrics Diagnosis & Treatment) 18^{th} ed., McGraw-Hill Medical (Sep. 25, 2006).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. Beneficial or desired clinical results include, but are not limited to, alleviation of one or more symptom(s), diminishment of extent of the deficit, stabilized (i.e., not worsening) state of cancer progression, delay or slowing of metastasis or invasiveness, and amelioration or palliation of symptoms associated with the cancer. Treatment also includes a decrease in mortality or an increase in the lifespan of a subject as compared to one not receiving the treatment.

As used herein, the term “administering,” refers to the placement an agent as disclosed herein into a subject by a method or route which results in at least partial localization of the agents at a desired site.

“A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. As used herein, the term “carcinoma” refers to a cancer arising from epithelial cells. As used herein, the term “invasive” refers to the ability to infiltrate and destroy surrounding tissue. Melanoma is an invasive form of skin tumor. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

As used herein, “Lyn” or “Lyn kinase” refers to Lyn tyrosine kinase.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. The term does not denote a particular age or sex. Thus, adult and newborn subjects, as well as fetuses, whether male or female, are intended to be included within the scope of this term.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as an chronic pancreatitis, fibrosis, autoimmune disease, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

“Therapeutically effective amount” as used herein refers to that amount which is capable of achieving beneficial results in a mammalian subject with fibrosis, chronic inflammation, chronic pancreatitis, cancer or inflammatory myofibroblastic tumors (IMTs). A therapeutically effective amount can be determined on an individual basis and will be based, at least in part, on consideration of the physiological characteristics of the mammal, the type of delivery system or therapeutic technique used and the time of administration relative to the progression of the disease.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to fibrosis, chronic inflammation, chronic pancreatitis, cancer or inflammatory myofibroblastic tumors (IMTs).

The terms “RNA interference agent” and “RNA interference” as they are used herein are intended to encompass those forms of gene silencing of Lyn kinase mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA). Accordingly, in some embodiments, a Lyn kinase inhibitor is a Lyn kinase RNA interference agent. In an exemplary embodiment, the RNAi agent is an siRNA obtained from Thermo Scientific, as described herein.

As used herein, “stellate cells” refer to fibroblastoid or fibroblast-like cells in, for example, pancreas and liver. The fibroblastoid or fibroblast-like cells are present in many organs and may be in a quiescent state or activated state.

As used herein “quiescent stellate cells” are fibroblastoid or fibroblast-like cells which, for example, produce collagen and form connective tissue important for normal organization of structure of the organs.

As used herein, “activated stellate cells” or “myofibroblasts” refer to stellate cells or precursors of myofibroblasts in other organs which are not quiescent, producing large and excessive amounts of collagen and inflammatory molecules as well as produce alpha-smooth muscle actin (α-SMA), proliferate and migrate. α-SMA is a marker for activated stellate cells in pancreas and liver and the activated myofibroblastic cells in other organs. Lyn kinase mediates activation of quiescent stellate cells and other precursors to their activated stellate cell of myofibroblastic state. There are several subclasses of myofibroblasts which are organ specific. In some embodiments, hyperactivity of Lyn kinase in activated stellate cells results in cancer. In some embodiments, hyperactivity of Lyn kinase in activated stellate cells results in fibrosis which may lead to cancer.

The term “agent” as used herein in reference to a Lyn kinase inhibitor means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In some embodiments, agents are small molecules having a chemical moiety. For example, chemical moieties include unsubstituted or substituted alkyl, aromatic, or heterocyclyl moieties. Compounds can be known to have a desired activity and/or property, e.g., inhibit Lyn kinase activity, or can be selected from a library of diverse compounds, using, for example, screening methods.

Chronic pancreatitis involves infiltration and replacement of normal parenchyma with stellate cells. The inventors have identified the hyperactive CXCR4/Lyn kinase chemotaxis signaling in stellate cells of chronic pancreatitis. CXCR4 receptor and Lyn kinase activation co-express with marker of activated stellate cells (a-smooth muscle actin), and are increased by 15-fold and 11-fold, respectively, in chronic pancreatitis versus normal. Lyn kinase inhibitors (siRNA or Bafetinib) prevent SDF-1/CXCR4-mediated migration of activated stellate cells, indicating that CXCR4/Lyn kinase can be a novel targetable pathway in human chronic pancreatitis, fibrosis, chronic inflammation and/or cancer (for example, cancers associated with chronic pancreatitis and/or fibrosing in an organ). Activated stellate cells or myofibroblasts in specific organs may cause fibrosis and/or cancer due of increased activity relative to normal subjects (hyperactive Lyn kinase). For example, hyperactivity of Lyn kinase in the activated stellate cells in the pancreas may result in pancreatitis, pancreatic fibrosis and pancreatic cancer. Similarly, activated stellated cells in the liver or myofibroblasts in lung or kidney for example, may result in fibrosis of the said organs which may lead to cancer.

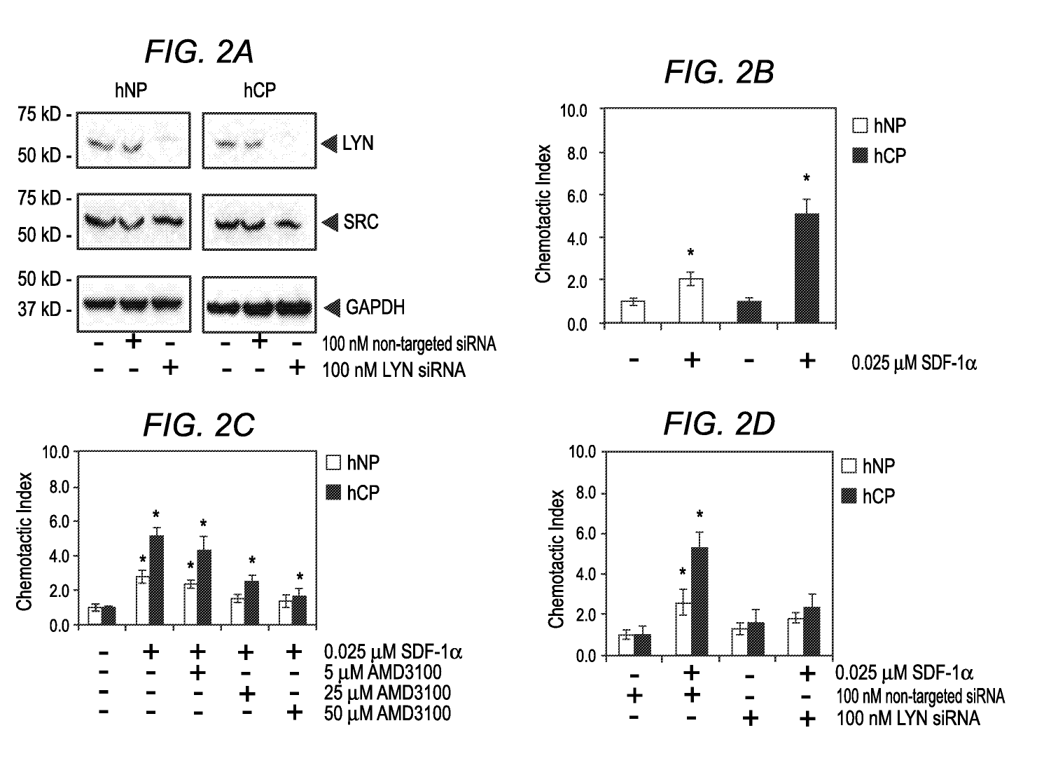

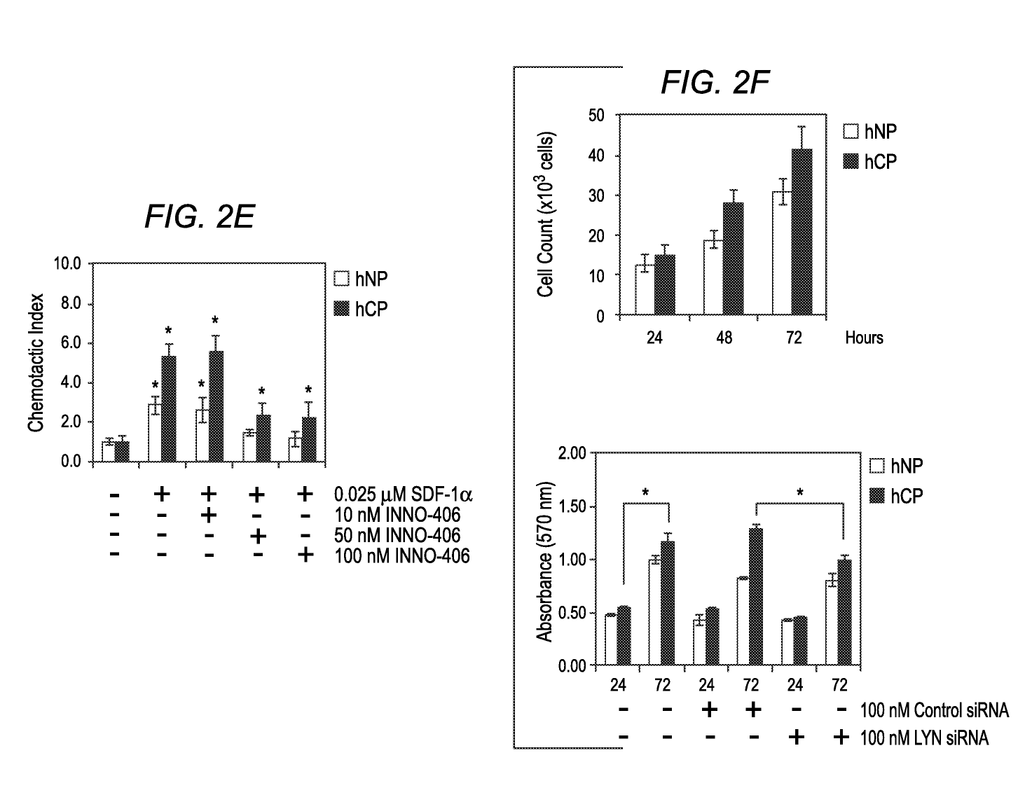

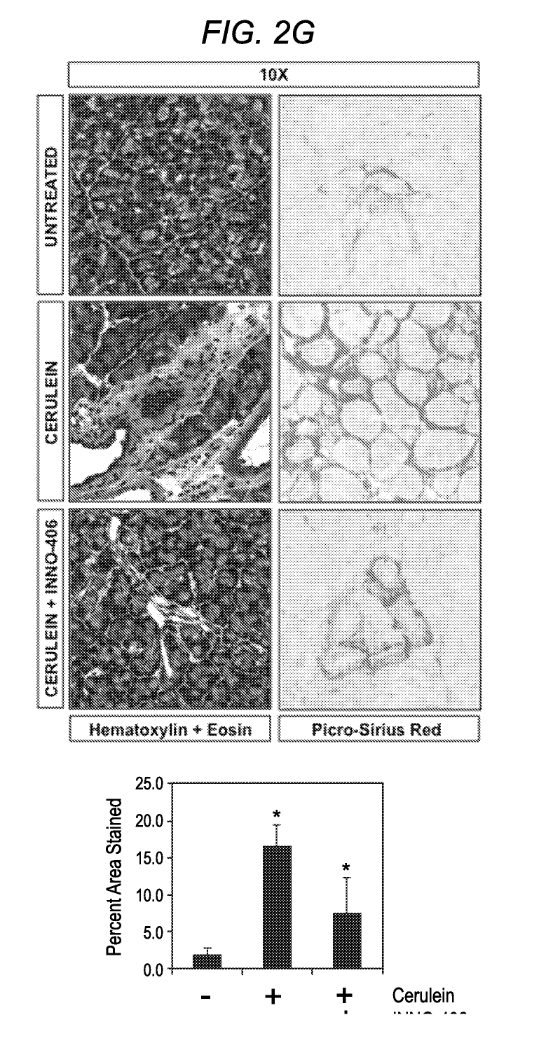

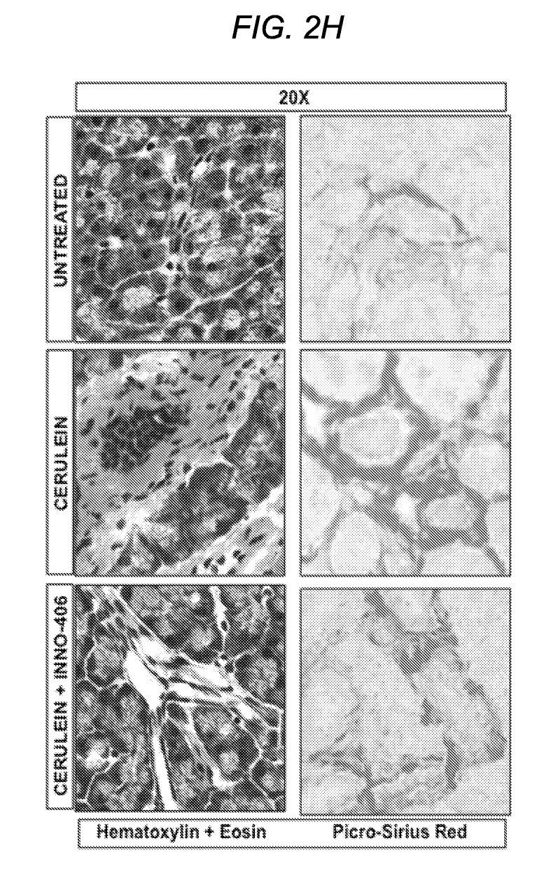

As shown herein, inhibition of Lyn kinase with inhibitors described herein (such as siRNA specific to Lyn kinase or Bafetinib), simultaneously blocks: (1) proliferation of activated pancreatic stellate cells (FIG. 2F); (2) migration of activated pancreatic stellate cells and 3) fibrosis (FIG. 2G). Thus, Lyn kinase is a positive regulator of activated stellate cell movement, proliferation and collagen production, and is a critical and unique therapeutic target in fibrosis and/or cancer, including but not limited to fibrosis and/or cancer of the pancreas, lung, liver or kidney.

### Methods

Accordingly, provided herein is a method for treating, inhibiting, reducing the severity of and/or promoting prophylaxis of a disease-state in a subject in need thereof. The method includes providing a composition comprising a Lyn kinase inhibitor and administering an effective amount of the composition to the subject so as to treat, inhibit, reduce the severity of and/or promote prophylaxis of the disease state. In various embodiments, the disease-state is fibrosis, chronic pancreatitis, chronic inflammation, cancer or inflammatory myofibroblastic tumors (IMTs). In some embodiments, the cancer is associated with chronic pancreatitis and/or fibrosis in an organ. In some embodiments, the Lyn kinase inhibitor is a siRNA specific for Lyn kinase. In an embodiment, the Lyn kinase inhibitor is Bafetinib. In one embodiment, the subject is human. In various embodiments, the composition is administrated to the subject before, during, or after the subject develops the disease-state. In some embodiments, the composition is administrated to the subject 1-3 times per day or 1-7 times per week. In some embodiments, the composition is administrated to the subject for 1-5 days, 1-5 weeks, 1-5 months, or 1-5 years. In various embodiments, the method further comprises treating the subject with surgery, radiation therapy, or chemotherapy, or a combination thereof. The surgery, radiation therapy, or chemotherapy, or a combination thereof may be provided before, during or after administering a therapeutically effective amount of the composition to the subject. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of chronic pancreatitis in a subject in need thereof. The method includes providing a composition comprising a Lyn kinase inhibitor and administering an effective amount of the composition to the subject so as to treat, inhibit, reduce the severity of and/or promote prophylaxis of chronic pancreatitis in the subject. In some embodiments, the Lyn kinase inhibitor is a siRNA specific for Lyn kinase. In an embodiment, the Lyn kinase inhibitor is Bafetinib. In one embodiment, the subject is human. In some embodiments, the methods may further comprise administering existing treatments for chronic pancreatitis and managing chronic pancreatitis using, for example, pharmacologic alleviation of abdominal pain, pharmacologic restoration of digestion and absorption, endoscopic treatments, surgical therapies, dietary care or combinations thereof. The composition comprising the Lyn kinase inhibitor and existing therapies may be administered sequentially or simultaneously. In some embodiments, the method comprises, consists of or consists essentially of providing a Lyn kinase inhibitor and administering an effective amount of the inhibitor to the subject.

Provided herein are methods for treating, inhibiting, reducing the severity of or promoting prophylaxis of fibrosis in a subject in need thereof. The method includes providing a composition comprising and Lyn kinase inhibitor and administering an effective amount of the composition to the subject so as to treat, inhibit, reduce the severity of and/or promote prophylaxis of fibrosis. In some embodiments, the Lyn kinase inhibitor is a siRNA specific for Lyn kinase. In an embodiment, the Lyn kinase inhibitor is Bafetinib. In one embodiment, the subject is human. In some embodiments, secondary organ fibrosis is a side-effect of radiation therapy. Accordingly, in one embodiment, the subject being treated for fibrosis using the Lyn kinase inhibitors is a subject that has undergone or is undergoing radiation therapy for a cancer in the first organ and shows or may show symptoms or other findings of fibrosis in a second organ, wherein the first and second organs are different. In some embodiments, the subject being treated for fibrosis is a subject that has undergone or is undergoing radiation therapy for treatment of cancer the first organ and shows symptoms or other findings of fibrosis at another location in the same organ. In some embodiments, the subject being treated for fibrosis is a subject undergoing radiation for treatment of cancer and shows or may show symptoms or other findings of fibrosis at the site of the cancer. In some embodiments, the subject being treated for fibrosis is a subject that has been exposed to radiation due to occupational reasons such as administrators of radiation therapy, military personnel and the like. The methods for treating, inhibiting, reducing the severity of or promoting prophylaxis of fibrosis may further comprise administering existing therapies for organ-specific fibrosis. In an exemplary embodiment, the methods may further comprise pharmacologic alleviation of abdominal pain, pharmacologic restoration of digestion and absorption, endoscopic treatments, surgical therapies, dietary care or combinations thereof for fibrosis in the pancreas. In an exemplary embodiment, the methods may further comprise oxygen therapy, prednisone, azathioprine and/or N-acetylcysteine for lung fibrosis. In another exemplary embodiment, the methods may further comprise administering angiotensin converting enzyme inhibitor or Lisinopril for myocardial fibrosis. The composition comprising the Lyn kinase inhibitor and existing therapies may be administered sequentially or simultaneously. In some embodiments, the method comprises, consists of or consists essentially of providing an agent that inhibits Lyn kinase and administering an effective amount of the agent to the subject.

In various embodiments of the methods described herein, fibrosis/fibrosing disorder is any one or more of fibrosis of the pancreas, cystic fibrosis, mediastinal fibrosis, myelofibrosis, retroperitoneal fibrosis, progressive massive fibrosis, nephrogenic systemic fibrosis, Crohn's Disease, Keloid, Scleroderma/systemic sclerosis, Arthrofibrosis, Peyronie's disease, Dupuytren's contracture, adhesive capsulitis, fibrosis of the liver, fibrosis of the lung, fibrosis of the intestine (for example, in Crohn's disease), fibrosis of the heart, or combinations thereof.

Provided herein are methods for treating, inhibiting, reducing the severity of or promoting prophylaxis of chronic inflammation in a subject in need thereof. The method includes providing a composition comprising and Lyn kinase inhibitor and administering an effective amount of the composition to the subject so as to treat, inhibit, reduce the severity of and/or promote prophylaxis of chronic inflammation in the subject. In some embodiments, the Lyn kinase inhibitor is a siRNA specific for Lyn kinase. In an embodiment, the Lyn kinase inhibitor is Bafetinib. In one embodiment, the subject is human. In some embodiments, the methods further comprise administering nonsteroidal anti-inflammatory drugs (NSAIDs). The composition comprising the Lyn kinase inhibitor and NSAIDs may be administered sequentially or simultaneously. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of disease-states associated with hyperactive Lyn kinase activity in activated stellate cells and other myofibroblastic cells. The method includes providing a composition comprising and Lyn kinase inhibitor and administering an effective amount of the composition to the subject so as to treat, inhibit, reduce the severity of and/or promote prophylaxis of disease-states associated with hyperactive Lyn kinase chemotaxis signaling in stellate cells. In some embodiments, the Lyn kinase inhibitor is a siRNA specific for Lyn kinase. In an embodiment, the Lyn kinase inhibitor is Bafetinib. In one embodiment, the subject is human. In exemplary embodiments, the disease-states include chronic pancreatitis, chronic inflammation, fibrosis, cancer or a combination thereof. In some embodiments, the method comprises, consists of or consists essentially of providing an agent that inhibits Lyn kinase and administering an effective amount of the agent to the subject.

Provided herein are methods for treating, inhibiting, reducing the severity of or promoting prophylaxis of disease-states associated with activated stellate cell proliferation and/or activated stellate cell migration. The method includes providing a composition comprising and Lyn kinase inhibitor and administering an effective amount of the composition to the subject so as to treat, inhibit, reduce the severity of and/or promote prophylaxis of disease-states associated with stellate cell proliferation and/or stellate cell migration. In some embodiments, the Lyn kinase inhibitor is a siRNA specific for Lyn kinase. In an embodiment, the Lyn kinase inhibitor is Bafetinib. In one embodiment, the subject is human. In exemplary embodiments, the disease-states include chronic pancreatitis, chronic inflammation, fibrosis, cancer or a combination thereof. In some embodiments, the method comprises, consists of or consists essentially of providing an agent that inhibits Lyn kinase and administering an effective amount of the agent to the subject.

Provided herein is a method for treating cancer or treating or preventing cancer metastasis in a subject in need thereof. The method includes providing a composition comprising and Lyn kinase inhibitor and administering an effective amount of the composition to the subject so as to prevent cancer metastasis in the subject. In some embodiments, the Lyn kinase inhibitor is a siRNA specific for Lyn kinase. In an embodiment, the Lyn kinase inhibitor is Bafetinib. In one embodiment, the subject is human. In some embodiments, the method comprises, consists of or consists essentially of providing an agent that inhibits Lyn kinase and administering an effective amount of the agent to the subject. In some embodiments, the cancer is associated with chronic pancreatitis and/or fibrosis in an organ. In various embodiments, the method further comprises treating the subject with surgery, radiation therapy, or chemotherapy, or a combination thereof. The surgery, radiation therapy, or chemotherapy, or a combination thereof may be provided before, during or after administering a therapeutically effective amount of the composition comprising the Lyn kinase inhibitor to the subject. In various embodiments of the methods described herein, the cancer is any one or more of lymphomas, sarcomas, brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer. In some embodiments, the cancer is associated fibrosis in the specific organ.

Provided herein are methods for treating, inhibiting, reducing the severity of or promoting prophylaxis of inflammatory myofibroblastic tumors in a subject in need thereof. The method includes providing a composition comprising and Lyn kinase inhibitor and administering an effective amount of the composition to the subject so as to treat, inhibit, reduce the severity of and/or promote prophylaxis of inflammatory myofibroblastic tumors in the subject. In some embodiments, the Lyn kinase inhibitor is a siRNA specific for Lyn kinase. In an embodiment, the Lyn kinase inhibitor is Bafetinib. In one embodiment, the subject is human. In some embodiments, the method comprises, consists of or consists essentially of providing an agent that inhibits Lyn kinase and administering an effective amount of the agent to the subject. In various embodiments of the methods described herein the inflammatory myofibroblastic tumors are in any one or more of bladder, bone, breast, CNS tumor, colon tumor, eye (orbit), heart tumor, kidney tumor, liver tumor, lung tumor, lymph node, mediastinum, pancreas, salivary glands, small bowel, spleen and/or thyroid gland. In some embodiments of the methods described herein the inflammatory myofibroblastic tumors are in any one or more of bladder, bone, breast, CNS tumor, colon tumor, eye (orbit), heart tumor, kidney tumor, liver tumor, lung tumor, lymph node, mediastinum, pancreas, salivary glands, small bowel, spleen and/or thyroid gland.

### Therapeutic Agents

In various embodiments, the agents that inhibit Lyn kinase or compositions comprising an agent that inhibits Lyn kinase may be administered concurrently or sequentially with other therapeutic agents including but not limited to chemotherapeutic agents, radiation therapy, immunotherapy agents or hormonal therapy agents.

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, therapies include, for example, radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or tele-therapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, therapies include, for example, immunotherapy. Immunotherapy may comprise, for example, use of cancer vaccines and/or sensitized antigen presenting cells. In some embodiments, therapies include targeting cells in the tumor microenvironment or targeting immune cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, therapies include, for example, hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In various embodiments, the compositions comprise Lyn kinase inhibitor is selected from the group consisting of a small molecule, a peptide, an antibody or a fragment thereof, a nucleic acid molecule and a bispecific polypeptide agent comprising binding sites specific for Lyn kinase and CXCR4. In some embodiments, the antibody is selected from the group consisting of monoclonal antibody or fragment thereof, a polyclonal antibody or a fragment thereof, chimeric antibodies, humanized antibodies, human antibodies, blocking or antagonistic antibody and a single chain antibody. In an embodiment, the Lyn kinase inhibitor is a siRNA molecule specific for Lyn kinase. In an embodiment, the Lyn kinase inhibitor is Bafetinib.

In some embodiments, provided herein are agents or compositions comprising agents for decreasing or inhibiting interactions between Lyn kinase and CXCR4. The compositions comprise agents that inhibit (reduce or block) signaling mediated by interaction between Lyn kinase and CXCR4. Such agents include, but are not limited to, antibodies (“antibodies” includes antigen-binding portions of antibodies such as epitope or antigen-binding peptides, paratopes, functional CDRs; recombinant antibodies; chimeric antibodies; tribodies; midibodies; or antigen-binding derivatives, analogs, variants, portions, or fragments thereof), protein-binding agents, small molecules, recombinant protein, peptides, aptamers, avimers and protein-binding derivatives, portions or fragments thereof.

Antisense oligonucleotides represent another class of agents that are useful in the compositions and methods described herein, particularly as Lyn kinase and/or CXCR4 antagonists. This class of agents and methods for preparing and using them are all well-known in the art, as are ribozyme and miRNA molecules. See, e.g., PCT US2007/024067 for a thorough discussion. Alternatively, an agent that inhibits interactions between Lyn kinase and CXCR4 can, in some embodiments of the compositions and methods described herein, include recombinant Lyn kinase or conjugates, or protein or antibody, small interfering RNA specific for or targeted to Lyn kinase mRNA, and antisense RNA that hybridizes with the mRNA of Lyn kinase, for example.

As used herein, a “blocking” antibody or an antibody “antagonist” is one which inhibits or reduces the biological activity of the antigen(s) it binds. For example, a Lyn kinase/CXCR4 bispecific blocking or antagonist antibody binds Lyn kinase and CXCR4 and inhibits the Lyn kinase/CXCR4 mediated chemotaxis signaling in stellate cells. In certain embodiments, the blocking antibodies or antagonist antibodies or portions thereof described herein completely inhibit the interaction between Lyn kinase and CXCR4. In certain embodiments, the blocking antibodies or antagonist antibodies or portions thereof described herein reduce/decrease the interaction between Lyn kinase and CXCR4. In an embodiment, the antibody is a monoclonal antibody that specifically binds Lyn kinase.

In some embodiments of the compositions and methods described herein, an agent that inhibits Lyn kinase is an RNA interference agent that specifically targets Lyn kinase and can be used for the inhibition of expression of Lyn kinase in vivo. RNA interference (RNAi) uses small interfering RNA (siRNA) duplexes that target the messenger RNA encoding a target polypeptide for selective degradation and is a powerful approach for inhibiting the expression of selected target polypeptides. siRNA-dependent post-transcriptional silencing of gene expression involves cleaving the target messenger RNA molecule at a site guided by the siRNA. “RNA interference (RNAi),” as used herein, refers to the evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In some embodiments, the RNA interference agent or siRNA is a double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes (for examples, genes encoding Lyn kinase). As used herein, “inhibition of target gene expression” includes any decrease in expression or protein activity or level of the target gene (for examples, genes encoding Lyn kinase) or protein (for examples, Lyn kinase) encoded by the target gene as compared to a situation wherein no RNA interference has been induced. The decrease will be of at least 10%, at least 20%, at least 30%, at least 40%, at least 50%, at least 60%, at least 70%, at least 80%, at least 90%, at least 95%, or at least 99% or more as compared to the expression of a target gene or the activity or level of the protein encoded by a target gene which has not been targeted by an RNA interfering agent.

As used herein, siRNAs also include small hairpin (also called stem loop) RNAs (shRNAs). In some embodiments, these shRNAs are composed of a short (e.g., about 19 to about 25 nucleotide) antisense strand, followed by a nucleotide loop of about 5 to about 9 nucleotides, and the analogous sense strand. Alternatively, in other embodiments, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent can be a cellular gene or genomic sequence, e.g., the human Lyn kinase genomic sequence. An siRNA can be substantially homologous to the target gene or genomic sequence, or a fragment thereof, i.e., the Lyn kinase gene or mRNA. As used in this context, the term “homologous” is defined as being substantially identical, sufficiently complementary, or similar to the target Lyn kinase mRNA, or a fragment thereof, to effect RNA interference of the target Lyn kinase. In addition to native RNA molecules, RNA suitable for inhibiting or interfering with the expression of a target sequence include RNA derivatives and analogs. Preferably, the siRNA is identical to its target. The siRNA preferably targets only one sequence.

Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one can also screen the potential target sequences for similar sequences in the sequence databases to identify potential sequences which may have off-target effects. For example, according to Jackson et al. (Id.), 15, or perhaps as few as 11 contiguous nucleotides, of sequence identity are sufficient to direct silencing of non-targeted transcripts. Therefore, one can initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human Lyn kinase mRNA.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence may be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated. The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA. The Examples herein provide specific examples of RNA interfering agents that effectively target Lyn kinase mRNA, including an siRNA (catalog no. L-003153-00-0010) purchased from Thermo Scientific., and assays for testing their Lyn kinase inhibitory activity.

In some embodiments, the RNA interference agent targeting Lyn kinase is delivered or administered in a pharmaceutically acceptable carrier. Additional carrier agents, such as liposomes, can be added to the pharmaceutically acceptable carrier. In another embodiment, the RNA interference agent is delivered by a vector encoding the small hairpin RNA (shRNA) in a pharmaceutically acceptable carrier to the cells in an organ of an individual. The shRNA is converted by the cells after transcription into siRNA capable of targeting Lyn kinase.

In some embodiments, the vector is one that can be regulated, such as tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, the RNA interference agents used in the methods described herein are taken up actively by cells in vivo following intravenous injection, e.g., hydrodynamic injection, without the use of a vector, illustrating efficient in vivo delivery of the RNA interfering agents. One method to deliver the siRNAs is catheterization of the blood supply vessel of the target organ. Other strategies for delivery of the RNA interference agents, e.g., the siRNAs or shRNAs used in the methods described herein, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs targeting Lyn kinase described herein, using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting Lyn kinase mRNA, can be delivered singly, or in combination with other RNA interference agents, e.g., siRNAs, such as, for example siRNAs directed to other cellular genes.

Synthetic siRNA molecules, including shRNA molecules, can be generated using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science , Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule for use in the compositions and methods described herein can be selected from a given target gene sequence, e.g., a Lyn kinase coding sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule for use in the compositions and methods described herein involves identifying a 23 nucleotide sequence motif AA(N19)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al., (2001) supra and Elbashir et al., 2001 supra). Analysis of sequence databases, including but not limited to the NCBI, BLAST, Derwent and GenSeq as well as commercially available oligosynthesis companies such as OLIGOENGINE®, can also be used to select siRNA sequences against EST libraries to ensure that only one gene is targeted.

### Delivery of RNA Interfering Agents

Methods of delivering RNA interference agents, e.g., an siRNA, or vectors containing an RNA interference agent, to the target cells, e.g., stellate cells or other desired target cells, for uptake include injection of a composition containing the RNA interference agent, e.g., an siRNA targeting Lyn kinase, or directly contacting the cell, e.g., a stellate cell, with a composition comprising an RNA interference agent, e.g., an siRNA targeting Lyn kinase. In other embodiments, an RNA interference agent, e.g., an siRNA targeting Lyn kinase, can be injected directly into any blood vessel, such as vein, artery, venule or arteriole, via, e.g., hydrodynamic injection or catheterization. Administration can be by a single injection or by two or more injections. The RNA interference agent is delivered in a pharmaceutically acceptable carrier. One or more RNA interference agents can be used simultaneously. In some preferred embodiments, only one siRNA that targets human Lyn kinase is used.

In some embodiments, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents targeting Lyn kinase, e.g., the siRNAs or shRNAs, can be introduced along with components that perform one or more of the following activities: enhance uptake of the RNA interfering agents, e.g., siRNA, by the cell, e.g., stellate cells or other cells; inhibit annealing of single strands; stabilize single strands; or otherwise facilitate delivery to the target cell and increase inhibition of the target Lyn kinase. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

In various embodiments, the agent that inhibits Lyn kinase or the composition comprising the Lyn kinase inhibitor for use with the methods described herein is administrated to the subject before, during, or after the subject develops the disease-state. In some embodiments, the composition is administrated to the subject 1-3 times per day or 1-7 times per week. In some embodiments, the composition is administrated to the subject for 1-5 days, 1-5 weeks, 1-5 months, or 1-5 years. In various embodiments, the disease-state is fibrosis, chronic inflammation, chronic pancreatitis, cancer or inflammatory myofibroblastic tumors (IMTs). In some embodiments, the Lyn kinase inhibitor is a siRNA specific for Lyn kinase or Bafetinib.

In various embodiments, the Lyn kinase inhibitor is administered intravenously, intramuscularly, intraperitoneally, orally or via inhalation.

In various embodiments, the effective amount of the Lyn kinase inhibitor is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages of an effective amount of a Lyn kinase inhibitor can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, such as biopsied malignant tumors, or the responses observed in the appropriate animal models. In various embodiments, the compositions of the invention comprising the Lyn kinase inhibitor may be administered once a day (SID/QD), twice a day (BID), three times a day (TID), four times a day (QID), or more, so as to administer an effective amount of the Lyn kinase inhibitor to the subject, where the effective amount is any one or more of the doses described herein.

In various embodiments, the subject is selected from the group consisting of human, non-human primate, monkey, ape, dog, cat, cow, horse, rabbit, mouse and rat.

### Pharmaceutical Compositions

In various embodiments, the present invention provides pharmaceutical compositions including a pharmaceutically acceptable excipient along with a therapeutically effective amount of a Lyn kinase inhibitor, such as Bafetinib, Lyn kinase-specific siRNA or a combination thereof. In various embodiments, the Lyn kinase inhibitor is a small molecule, a peptide, a protein, an aptamer, an antibody or a fragment thereof, a nucleic acid molecule, or a bispecific polypeptide agent comprising binding sites specific for Lyn kinase and CXCR4

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins P A, USA) (2000).

In various embodiments, the composition is administrated to the subject 1-3 times per day or 1-7 times per week. In various embodiments, the composition is administrated to the subject for 1-5 days, 1-5 weeks, 1-5 months, or 1-5 years.

### Kits

The invention also provides a kit for the treatment of cancer and/or fibrosing disorder, inhibition of cancer and/or fibrosing disorder, reduction of cancer and/or fibrosing disorder or promotion of cancer and/or fibrosing disorder prophylaxis in a subject in need thereof. The kit comprises a composition comprising a Lyn kinase inhibitor and instructions for use of the composition for treating, inhibiting and/or reducing the severity of cancer and/or fibrosing disorder in subjects in need thereof. In some embodiments, the Lyn kinase inhibitor is a small molecule, a peptide, a protein, an aptamer, an antibody or a fragment thereof, a nucleic acid molecule or a bispecific polypeptide agent comprising binding sites specific for Lyn kinase and CXCR4.

The kit is an assemblage of materials or components, including at least one of the compositions described herein. Thus, in some embodiments the kit contains a composition including a Lyn kinase inhibitor, wherein the Lyn kinase inhibitor is a siRNA molecule specific for Lyn kinase or Bafetinib, as described herein.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat, reduce the severity of, inhibit or prevent cancer and/or fibrosing disorders in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing a Lyn kinase inhibitor. The packaging material generally has an external label which indicates the contents and/or purpose of the kit and/or its components.

### Screening Methods

Also provided herein are methods for identifying inhibitors of Lyn kinase. The methods comprise contacting the Lyn kinase in Lyn kinase positive cells with a molecule of interest and determining whether the contact results in decreased migration of activated stellate cells or myofibroblast, a decrease in stellate cell migration or myofibroblast being indicative that the molecule of interest is a Lyn kinase inhibitor. In another embodiment, decrease in the amount of Lyn kinase synthesized is indicative that the molecule of interest is inhibitor of Lyn kinase. In a further embodiment, decrease in the amount of nucleic acid (for example, mRNA) encoding Lyn kinase is indicative that the molecule of interest is an inhibitor of Lyn kinase. In some embodiments, the inhibitor inhibits the tyrosine kinase activity of Lyn kinase. In some embodiments, the inhibitor reduces or inhibits the synthesis of Lyn kinase. In an exemplary embodiment, the activated stellate cells are activated pancreatic stellate cells.

In various embodiments, the Lyn kinase inhibitor is selected from the group consisting of a small molecule, a peptide, an antibody or a fragment thereof, a bispecific antibody having an antigen binding site specific for Lyn kinase and CXCR4 and a nucleic acid molecule. In some embodiments, the screening method comprises separately contacting each of a plurality of samples to be tested. In some embodiments, the plurality of samples comprises more than about 10⁴ samples. In some embodiments, the plurality of samples comprises more than about 5×10⁴ samples. In some embodiments, the stellate cells are activated stellate cells. In some embodiments, the activated stellate cells are obtained from patients with chronic pancreatitis. In some embodiments, the activated stellate cells are obtained from patients with pancreatic cancer. In some embodiments, myofibroblasts are obtained from human tissues of a fibrosing disorder are used. In some embodiments, myofibroblasts are obtained from human cancers.

The compound of interest that inhibits Lyn kinase may be any one or more of a small molecule, a peptide, a protein, an aptamer, an antibody or a fragment thereof, a nucleic acid molecule, a bispecific polypeptide agent comprising binding sites specific for Lyn kinase and CXCR4.

Assays that may be employed to identify compounds that inhibit Lyn kinase include but are not limited to microarray assay, quantitative PCR, Northern blot assay, Southern blot assay, Western blot assay immunohistochemical assays, binding assays, gel retardation assays or assays using yeast two-hybrid systems. A person skilled in the art can readily employ numerous techniques known in the art to determine whether a particular agent inhibits Lyn kinase.

In some embodiments, the screening method comprises separately contacting each of a plurality of samples to be tested. In some embodiments, the plurality of samples comprises more than about 10⁴ samples. In some embodiments, the plurality of samples comprises more than about 5×10⁴ samples. In some embodiments, the stellate cells are activated stellate cells. In some embodiments, the activated stellate cells are obtained from patients with chronic pancreatitis.

### EXAMPLES

### Example 1

### Human Pancreatic Stellate Cells

Human stellate cells were examined using freshly harvested pancreatic tissue or freshly isolated primary human stellate cells in culture. Surgical pancreatic samples were obtained from 10 chronic pancreatitis patients, or donors without any pancreatic disease, using guidelines approved by the Institutional Review Board of the Cedars-Sinai Medical Center, Los Angeles, and University of Washington, Seattle.

Isolation of Stellate Cells from Human Pancreatic Tissue

Stellate cells were isolated from human pancreatic tissue (by using the tissue digestion method with pronase, collagenase, and DNAse), identified, and expanded in culture, using the same methods as described in detail by Apte et al. for rat pancreatic tissue (Apte, M V. et al. Gut., 43, 128-133 (1998). In our experiments, we used the early passage cells to minimize any potential effect of prolonged culturing on primary cells. The passage number of chronic pancreatitis or normal cells was in the range of 1 to 3, and the passage number was always identical for chronic pancreatitis and normal cells during each set of experiment.

At 24 hours in culture at least 95% of cells had attached to the wells and had assumed a stellate, angular appearance with prominent lipid droplets in the cytoplasm. According to this particular method, purity of the preparation was ultimately ˜100% (after a few passages in tissue culture) as assessed by vitamin A autofluorescence in quiescent stellate cells, and α-SMA in activated stellate cells (myofibroblasts).

### Immunofluorescence (Including Morphometric and Statistical Analysis) in Human Pancreatic Tissue

Immunolocalization of CXCR4 and α-SMA in pancreatic tissue specimens from cases of pancreatitis and from normal pancreas was performed following a method of indirect immunofluorescence with modifications (Cirulli, V. et al. J. Cell Biol., 150, 1445-1459 (2000); Yebra, M. et al. Developmental Cell, 5, 695-707 (2003); Diaferia, G R. et al. Development, 140, 3360-3372 (2013)). Briefly, two and three color immunofluorescence was performed on paraffin sections using standard procedures of deparaffinization followed by antigen retrieval, blocking of aldehyde residues with glycine, and incubation overnight with primary antibodies. Primary antibodies used were: rabbit anti-CXCR4 (Cat #ab2074, Abcam, Cambridge, Mass.), mouse anti-CXCR4 (Cat #ab58176, Abcam), mouse anti-α-SMA (Cat #A2547, Sigma, St. Louis, Mo.). After primary antibody reaction, sections were challenged with species-specific fluorophore-labeled F(ab)2 secondary antibodies LRSC-donkey anti-rabbit and FITC-donkey anti-mouse (Cat #ab150103, Abcam). After staining, sections were mounted with DAPI mounting medium to visualize nuclei, and analyzed on a NIKON Eclipse 90i microscope, equipped with a CoolSNAP-HQ2 CCD camera (Photometrics). Extensive morphometric analysis was performed on at least 30 sections per tissue samples, collected from cases of pancreatitis and normal pancreata, using the NIS Elements 3.22 software (Nikon). Collectively, morphometric analysis was performed on 150 images per tissue group (i.e. pancreatitis or normal pancreas) collected from a total of 180 tissue sections. Stained sections were then viewed on a Nikon Eclipse 90i, and morphometric measurement performed using Nikon Pro Plus software (Media Cybernetics, Inc.). Statistics. Statistical significance of differences in data values was validated by analysis of variance (ANOVA), followed by Bonferroni's Multiple Comparison Test, or by two tailed student's t test, using the Prism-4 statistical package (Graph Pad Software, San Diego, Calif.), with significance limit set at p<0.05.

### Immune Complex Kinase Assay

Preliminary qPCR experiments determined that only two members of the Src family, Src and Lyn, were substantially expressed in normal and CP human pancreatic stellate cells. Thus, we performed in vitro kinase assays on Src or Lyn immunoprecipitates (Ptasznik, A. et al. J Biol Chem., 270, 19969-19973 (1995)) to measure autophosphorylation (phosphorylation of the kinase by itself sustains its activated state and leads to an increase in enzymatic activity). Immunoprecipitation. Aliquots of stellate cells from CP or normal subjects were normalized for cell counts (3.5 min/assay), washed in 1× PBS, and lysed in ice-cold lysis buffer (2% NP-40, 10 mM Tris, 50 mM NaCl, 30 mM sodium pyrophosphate, 50 mM NaF) supplemented with protease inhibitor mixture as described5. Protein concentrations on whole cell lysates were determined by BCA protein assay. For immunoprecipitation, equal amounts of cell lysates were incubated at 4° C. with 5 μg of Lyn or Src Ab for 4 h followed by 30 μl of Pansorbin beads for 2 h. Immunoblot analysis of Lyn, Src, α-SMA, CXCR4 and GAPDH expression, was conducted on immunoprecipitates generated as described above, or directly on cell lysates containing 30 μg of protein. Kinase assay. Lyn or SRC immunoprecipitates were washed twice with lysis buffer (see above), and once with a buffer containing 10 mM Tris (pH 7.1), 100 mM NaCl, and 100 μM sodium orthovanadate. Kinase assays were performed by resuspending Lyn or SRC immunoprecipitates in 30 μl kinase reaction mixture (25 mM HEPES [pH 7.1], 10 mM MnCl2, 10 μCi [32P-γ]-ATP, 1 μM unlabeled ATP). Kinase reaction was carried out for 2 min at 20° C., and then the reaction was stopped by the addition of 30 μl sodium dodecyl sulfate (SDS) gel-loading buffer and boiling, resolved by 8% SDS-PAGE, and visualized by autoradiography.

siRNA

Four short interfering siRNAs targeting human Lyn and four non-targeting siRNAs (control) combined into one pool (Thermo Scientific, catalog #L-003153-00-0010 and D-001210-01-20, respectively) were prepared and stored frozen in aliquots at −20° C., as described in Ptasznik, A. et al. Nat Med. 10, 1187-119 (2004). Preparation of primary fibroblast-like stellate cells before electroporation as well as electroporation with siRNAs were performed according to the manufacturer's recommendations (see the Amaxa 4D-Nucleofector Basic Protocol for Primary Mammalian Fibroblasts and Amaxa 4D-Nucleofector Optimization Protocol for Primary Cells, online).

Transwell Migration Assay with SDF-1 in siRNA, AMD3100, and INNO-406-Treated Cells

SDF-1α was obtained from PeproTech Inc. and AMD3100 from Sigma-Aldrich (St. Louis, Mo.). INNO-406 was synthetized and purified at Cellagen Technology (San Diego, Calif.). The migration assays were performed by using transwell migration assays with polycarbonate membrane inserts with 8 μm pore size that is optimal for fibroblast-like cell migration (Corning Incorporated, NY, cat #3422). Stellate cells were treated with siRNA (100 nM) for 48 h, or with increasing concentrations of AMD3100 (5 μM-50 μM) or INNO-406 (10 nM-100 nM) for 60 minutes. Then, cells were placed in the migration upper chambers, in the presence or absence of SDF-1 (0.025 μM) with 2% FBS in the lower chambers. After 2 h of incubation, cells migrated to the lower chamber were counted, and the chemotactic index was determined as follows:

(number of cells migrating to SDF-1 chemokine)/(number of cells migrating to medium alone)

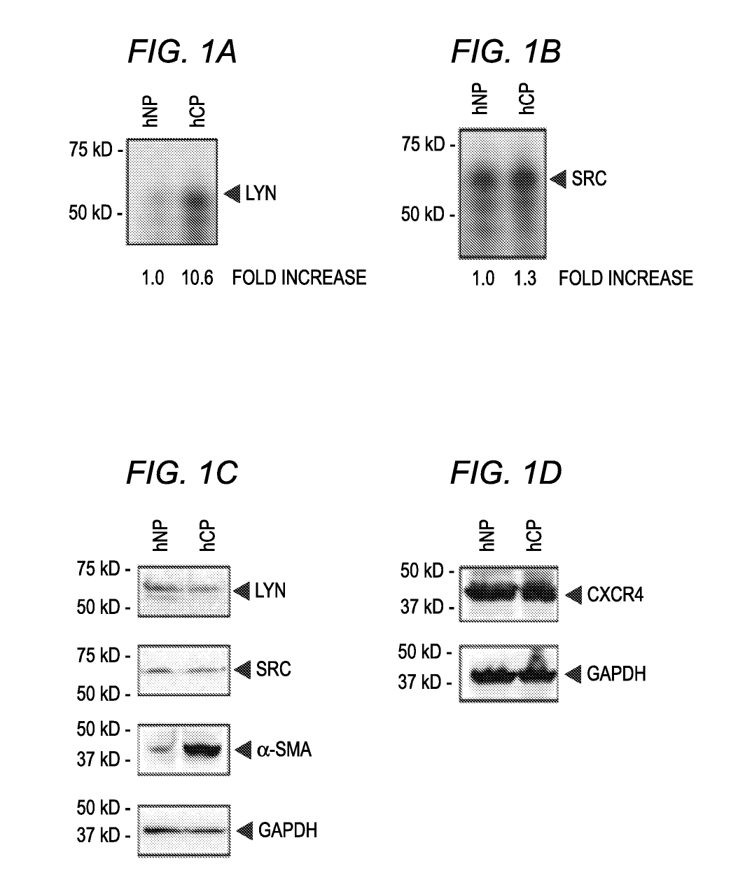

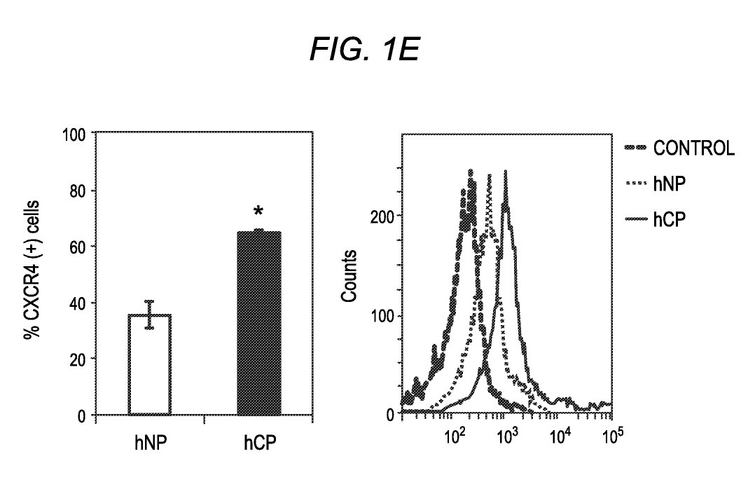

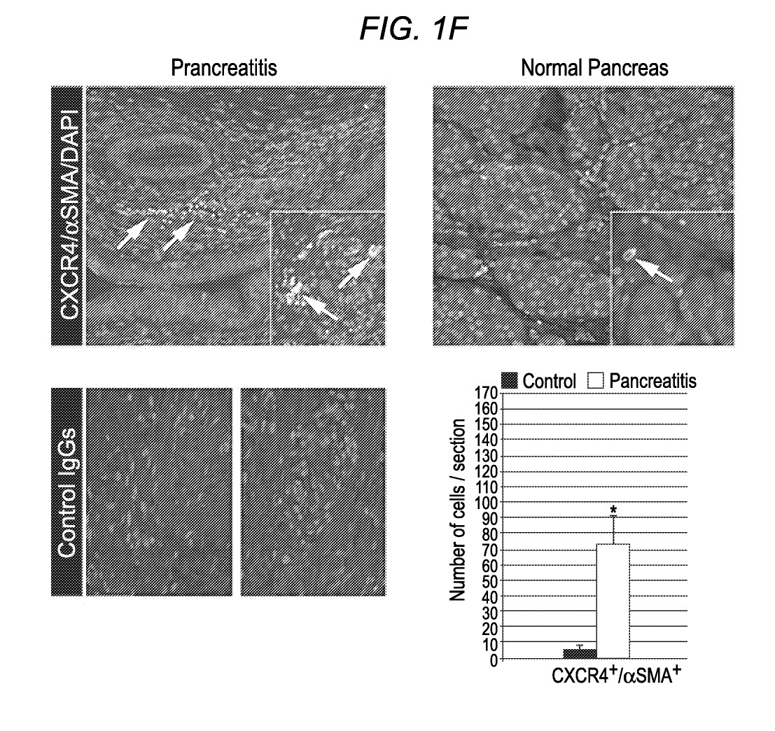

To distinguish chemotaxis versus chemokinesis, a control checkerboard analysis was performed. The data indicated that the cells migrated in greatest numbers when attractant (SDF-1) was added only to lower chamber (directed migration). However, cells (particularly chronic pancreatitis cells—hCP) were also observed to migrate in response to SDF-1 in the absence of established gradient (increased random migration). Thus, we detected that both chemotaxis (directional movement towards the SDF-1 chemokine gradient) and chemokinesis (nondirectional movement caused by the presence of SDF-1 chemokine) were significantly increased in chronic pancreatitis stellate cells (hCP), as compared to normal stellate cells (hNP), consistent with the hyperactive CXCR4/Lyn signaling in chronic pancreatitis stellate cells (FIG. 1a, 1e, 1f). Consequently, in the absence of SDF-1, there was no difference in spontaneous stellate cell migration between the chronic pancreatitis stellate cells and normal stellate cells.

### Viability/Proliferation Measurements

To be certain that unintended cytotoxic effects were not responsible for the observed decreases in stellate cell migration, in siRNA or AMD3100 or INNO-406-treated cells, control viability measurements were carried out by trypan blue exclusion assay and MTT assay, during the course of these experiments. As the positive controls for our viability measurements we used stellate cells deprived serum for 24-48 h. We consistently observed that stellate cells isolated from patients with chronic pancreatitis (hCP) had higher proliferation/viability rate than cells isolated from normal donors (hNP), and Lyn-dependent viability was increased in hCP as compared to hNP. The stellate cell accumulation problem in chronic pancreatitis is a balance equation where:

rate of stellate cell accumulation=rate of stellate cell proliferation−rate of stellate cell death

Therefore, we believe that MTT viability assay is the most proper to use in this particular case.

### In Vivo Effects of INNO-406 on Chronic Pancreatitis-Induced Fibrosis in Mouse Model

There is no optimal animal model available for human pancreatitis-induced fibrosis, to date. However, a most commonly used experimental model of chronic pancreatitis (CP) partially recapitulating human disease is repeated injection of cerulein into mice (Ulmasov, B. et al. Am J Pathol. 183, 692-708 (2013)). C57BL/6 is the most frequently used mouse strain for this type of biomedical research. In our current experiments, C57BL/6J mice (2 months old male and female) were divided into 3 groups (each group consisted of 15 animals) as follows: (1) untreated mice (saline only); (2) mice treated with cerulein for 6 weeks (50 μg/kg hourly, 6 hourly injections in one day constituted one treatment, twice-weekly); (3) mice treated with cerulein (as above) plus 120 mg INNO-406/kg per day for 6 weeks (oral gavage). When 120 mg INNO-406/kg per day is administrated orally to the mice, the concentration of INNO-406 in the soft tissues is estimated to be 0.24 μM according to pharmacokinetic studies described previously (Kimura, S. et al. Blood., 106, 3948-3954 (2005); Yokota, A, et al. Blood, 109, 306-314 (2007)). To allow resolution of acute inflammatory changes (in order to make fibrosis clearly visible and detectable), all mice were euthanized at 5 days after their final cerulein and INNO-406 treatment.

### Histological Quantitative and Statistical Analysis of Mice Pancreata

After treatment, pancreata from euthanized C57BL/6J mice were washed on ice with ice-cold PBS. Tissue were embedded in OCT compound (Tissue-Tek, Sakura Finetek, Torrance, Calif.) and quickly frozen using liquid nitrogen. Frozen blocks were submitted to Cedars-Sinai Biobank and Translational Research Core for sectioning and mounting on Superfrost slides (Fisher Scientific) as well as for hematoxylin & eosin (H&E) staining. For picro-sirius red staining, one set of frozen tissue slides were fixed for 10 min in acetone chilled at −20° C. and allowed to air dry to room temperature for 30 min. Slides were hydrated in PBS for 5 min, stained with picro sirius red solution for 30 min (Picro Sirius Red Stain kit, Cat #ab150681, Abcam, Cambridge, Mass.) and washed twice with 0.5% acetic acid (provided in the kit). The slides were dehydrated with 3 washes in absolute ethanol prior to coverslip mounting with mounting media (Acrymount, Cat #SL80-4, StatLab, McKinney, Tex.). All slides were digitally scanned at 20× on an Aperio ScanScopAT Turbo (Leica, Bufalo Grove, Ill.) and stored on Cedars-Sinai server by the Cedars-Sinai Confocal Microscopy Core Facility. Picro sirius red quantitation were performed using Leica Biosystem Tissue IA Optimizer web-based program that measures positive stained intensity per area at 10× magnification. Positive stained area is defined by intensity above background set for each slide. For each slide, 60 random, non-overlapping areas were selected and averaged as a percentage (positive stained area (μm2) per total area examined (μm2)). A two-tailed t-test was performed between untreated and cerulein-treated, untreated and cerulein/INNO-406-treated and cerulein-treated and cerulein/INNO-406-treated with a statistical set at p<0.05.

### Example 2

We began by screening for the expression and activities of known Src kinases in stellate cells isolated from pancreata of donors with or without CP (FIG. 1). After isolation of stellate cells from human pancreatic tissues kinase assays showed activities in Lyn and Src immunoprecipitates. We detected a ˜11 fold increase in Lyn tyrosine kinase activity in cells isolated from CP compared to cells from normal subjects (FIG. 1A). Importantly, we found no increase in Src kinase activity (a kinase of similar structure and function to Lyn) in CP stellate cells, as compared to normal cells (FIG. 1B). Thus, increase of Lyn activity in stellate cells of CP was selective. There was no change in the levels of Lyn or Src proteins, as determined by Western blotting (FIG. 1C). Importantly, α-SMA, a marker of activation of stellate cells as we described (Omary, M B. et al. J Clin Invest. 17, 50-59 (2007)), was increased in stellate cells from subjects with CP compared to those from normal subjects (FIG. 1C). These data indicated that increased Lyn kinase activity was associated with activation of stellate cells in CP patients.

Next, we compared the expression of CXCR4 in stellate cells isolated from CP patients and normal subjects, in cultured primary human cells and human pancreatic sections. Western blot analysis of total proteins, normalized to the housekeeping gene GAPDH, showed no differences in the total (surface+intracellular) cellular expression of CXCR4 in stellate cells isolated from CP patients and normal donors (FIG. 1D). In contrast, as indicated by flow cytometry analysis, the mean percentages of stellate cells expressing surface CXCR4 were significantly increased in CP, as compared to normal cells (FIG. 1E). Our results indicated that the number of CXCR4-positive stellate cells, infiltrating the pancreatic parenchyma, was dramatically increased in CP compared to normal pancreas.

The differential kinase activity of Lyn in CP stellate cells versus normal stellate cells (FIG. 1A) supports choosing Lyn as a therapeutic target. We silenced Lyn expression in CP patient- and normal donor-derived stellate cells, respectively, using siRNA (Ptasznik, A. et al. Nat Med. 10, 1187-1189 (2004)). After treatment of the cells Western blotting showed a ˜80-95% reduction in Lyn protein (FIG. 2A). Importantly, we found no inhibitory effect on Src, another Src family kinase of similar structure and size (FIG. 2A). Thus, the inhibition of Lyn by siRNA was both robust and selective. We also employed a mechanistically distinct approach to inhibit Lyn in stellate cells by using Lyn tyrosine kinase inhibitor INNO-406 (NS-187, Befetinib), as described. INNO-406 was proven an effective Lyn inhibitor at clinically relevant concentrations without affecting the phosphorylation of Src and several other Src family members (Kimura, S. et al. Blood., 106, 3948-3954 (2005)).

We then compared the SDF-1/CXCR4-induced chemotactic response of CP and normal stellate cells. Increase in the chemotactic index toward SDF-1 gradient was observed both in CP and normal cells (FIG. 2B), which could be blocked by treatment with AMD3100, a specific antagonist for the SDF-1 receptor CXCR4 (FIG. 2C). However, CP cells showed increased CXCR4-induced chemotaxis by ˜3-fold as compared to normal cells (FIG. 2B). The selective ablation of Lyn by siRNA (FIG. 2A) effectively inhibited chemotaxis of CP stellate cells (FIG. 2D). We also observed strong inhibition of chemotaxis following treatment with INNO-406 (FIG. 2E). Notably, inhibition of chemotaxis by Lyn inhibitors (siRNA or INNO-406) was much stronger in stellate CP cells than in normal cells, consistent with differential Lyn kinase activity (FIG. 1A). In addition, the selective ablation of Lyn by siRNA (FIG. 2A) effectively inhibited proliferation of CP stellate cells at 72 h (as compared to control siRNA), while leaving proliferation of normal stellate cells not affected, as measured by MTT assay (FIG. 2F). Together, these findings indicated that Lyn stimulated both chemotaxis and proliferation of stellate cells (this function of Lyn was previously established in other cellular systems Ptasznik, A. et al. J Exp Med. 196, 667-678 (2002); O'Laughlin-Brunner, B. et al. Blood. 98, 343-350 (2001)), and Lyn-mediated chemotaxis and proliferation were increased in stellate cells in CP, consistent with hyperactive Lyn kinase in CP (FIG. 1A).

Our finding with human primary stellate cells support targeting CXCR4/Lyn in CP patients by showing that CXCR4 receptor and Lyn activity are differentially expressed in stellate cells of CP compared to normal subjects, and inhibition of Lyn leads to the strong chemotaxis and proliferation blockade of primary CP stellate cells in vitro, while leaving chemotaxis and proliferation of normal stellate cells only slightly affected. However, it is known that selective blockade of one of the many chemokine receptors can be easy bypassed in vivo with the other inflammatory chemokines and their receptors, in chronic inflammation, including CP. Thus, this kind of treatment is usually not effective. This is important because the mean percentages of stellate cells expressing surface CCR5 (in addition to CXCR4) were significantly increased in CP, as compared to normal stellate cells. In contrast, inhibition of Lyn would not likely be bypassed in vivo, because Lyn is a common downstream target of several key pro-inflammatory chemoattractant receptors, including CXCR4, CCR5, and fMLP receptor (Ptasznik, A. et al. J Biol Chem. 270, 19969-19973 (1995); Ptasznik, A. et al. J Exp Med. 196, 667-678 (2002); Malik, M. et al. J Immunol. 181, 4632-4637 (2008); Tomkowicz, B. et al. Blood. 108, 1145-1150 (2006)). Thus, inhibition of Lyn prevents the chemotactic and proliferative action of several receptors (Ptasznik, A. et al. J Biol Chem. 270, 19969-19973 (1995); Ptasznik, A. et al. J Exp Med. 196, 667-678 (2002); Malik, M. et al. J Immunol. 181, 4632-4637 (2008); Tomkowicz, B. et al. Blood. 108, 1145-1150 (2006); O'Laughlin-Brunner, B. et al. Blood. 98, 343-350 (2001); Nakata, Y. et al. Blood. 107, 4234-4239. (2006)).

We therefore performed in vivo validation of Lyn as a potential therapeutic target in pancreatic fibrosis. We compared the pancreatic fibrosis in mice treated with cerulein, or both cerulein and INNO-406, for 6 weeks. Thereafter mice were killed and pancreata were subjected for histological analysis. Hematoxilin/Eosin and Picro-Sirius Red stains visualized the collagen and severe fibrosis in chronic pancreatitis mice that were treated with cerulein alone (FIG. 2G). Notably, the extent of fibrosis was significantly reduced in mice treated for 6 weeks with both cerulein and INNO-406 (FIG. 2G, FIG. 2H). These data indicated that the inhibitor of Lyn kinase, INNO-406, ameliorates cerulein-induced pancreatic fibrosis in vivo. We conclude that Lyn is an emerging relevant target in anti-fibrotic therapeutic strategies, and the inhibition of Lyn can prevent the replacement of normal parenchyma with fibrotic tissue in pancreas, and perhaps in liver, lung and other organs.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for treating, inhibiting, reducing the severity of and/or promoting prophylaxis of a disease-state in a subject in need thereof comprising:
(i) providing a composition comprising a Lyn inhibitor; and
(ii) administering a therapeutically effective amount of the composition to the subject so as to treat the disease-state in the subject.

2. The method of claim 1, wherein the disease-state is associates with increased Lyn-kinase activity in the subject relative to a normal subject.

3. The method of claim 2, wherein the increased Lyn-kinase activity is in activated stellate cells.

4. The method of claim 1, wherein the disease-state is fibrosis, chronic inflammation, chronic pancreatitis, pancreatic fibrosis, cancer or inflammatory myofibroblastic tumors (IMTs).

5. The method of claim 4, wherein the cancer is pancreatic cancer and is associated with chronic pancreatitis and/or pancreatic fibrosis.

6. The method of claim 1, wherein the cancer is associated with activated stellate cells or myofibroblasts.

7. The method of claim 4, wherein the inflammatory myofibroblastic tumors are in any one or more of bladder, bone, breast, CNS tumor, colon tumor, eye (orbit), heart tumor, kidney tumor, liver tumor, lung tumor, lymph node, mediastinum, pancreas, salivary glands, small bowel, spleen and/or thyroid gland.

8. The method of claim 4, wherein fibrosis is any one or more of mediastinal fibrosis, myelofibrosis, retroperitoneal fibrosis, progressive massive fibrosis, nephrogenic systemic fibrosis, Crohn's Disease, Keloid, Scleroderma/systemic sclerosis, Arthrofibrosis, Peyronie's disease, Dupuytren's contracture, adhesive capsulitis, fibrosis of the liver, fibrosis of the lung, fibrosis of the pancreases, fibrosis of the intestine, Crohn's disease, fibrosis of the heart, fibrosis resulting from radiation therapy, fibrosis resulting from occupational exposure to radiation or combinations thereof.

9. The method of claim 4, wherein the cancer is any one or more of lymphomas, sarcomas, brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer.

10. A method for preventing cancer metastasis by the method of claim 1.

11. The method of claim 1, wherein the Lyn inhibitor is selected from the group consisting of a small molecule, a peptide, a protein, an aptamer, an antibody or a fragment thereof, a nucleic acid molecule, a bispecific polypeptide agent comprising binding sites specific for Lyn and CXCR4.

12. The method of claim 9, wherein the bispecific polypeptide comprises an antibody or antigen binding portion thereof that specifically binds Lyn and an antibody or antigen binding portion thereof that specifically binds CXCR4.

13. The method of claim 9, wherein the nucleic acid molecule is a siRNA molecule of Lyn.

14. The method of claim 9, wherein the antibody is selected from the group consisting of monoclonal antibody or fragment thereof, a polyclonal antibody or a fragment thereof, chimeric antibodies, humanized antibodies, human antibodies, antagonistic antibody, bispecific antibody and a single chain antibody.

15. The method of claim 9, wherein the Lyn inhibitor is Bafetinib.

16. The method of claim 1, wherein the composition is administered intravenously, intramuscularly, intraperitoneally, orally or via inhalation.

17. The method of claim 1, wherein the effective amount of the Lyn inhibitor is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

18. The method of claim 1, wherein the subject is human.

19. The method of claim 1, wherein the composition is administrated to the subject before, during, or after the subject develops the disease-state.

20. The method of claim 1, wherein the composition is administrated to the subject 1-3 times per day or 1-7 times per week.

21. The method of claim 1, wherein the composition is administrated to the subject for 1-5 days, 1-5 weeks, 1-5 months, or 1-5 years.

22. The method of claim 1, further comprising treating the subject with surgery, radiation therapy, chemotherapy or a combination thereof.

23-32. (canceled)
